(19) United States
(12) Patent Application Publication
(10) Pub. No.: US 2010/0183730 A1
(43) Pub. Date: Jul. 22, 2010

(54) High dose composition of ursodeoxycholic acid

(76) Inventors: Johannes Jan Platteeuw, Boxtel (NL); Hiteshkumar Anilkant Doshi, Mumbai (IN)

Correspondence Address: BROWDY AND NEIMARK, P.L.L.C.;624 NINTH STREET, NW, SUITE 300, WASHINGTON, DC, 20001-5303, US

(21) Appl. No.: 12/596,506

(22) PCT Filed: Apr. 18, 2008
(86) PCT No.: PCT/NL08/50229

§ 371 Date: Apr. 1, 2010

(30) Foreign Application Priority Data
Apr. 19, 2007 (EP) 07106513.0

(51) Int. Cl. A61K 9/14; A61K 31/575; A61P 1/16

(52) U.S. Cl. 424/495; 424/490; 514/182; 424/494; 424/497

(57) ABSTRACT

The present invention relates to high dose multiparticulate pharmaceutical formulation comprising ursodeoxycholic acid for oral administration, as well as a method for preparing said composition.

### FIELD OF THE INVENTION

The present invention concerns a pharmaceutical formulation comprising a high dose composition of ursodeoxycholic acid.

In particular, it concerns an immediate release formulation containing a high dose of a multiparticulate with ursodeoxycholic acid for oral administration, as well as a method for preparing said composition.

### BACKGROUND OF THE INVENTION

Ursodeoxycholic acid (UDCA) is a bile acid and has since long been used in the dissolution of cholesterol gall bladder stones, and this is still the primary indication. Later on it was demonstrated that its long-term use is safe and is effective in treating patients suffering from primary biliary cirrhosis (PBC). Since then, a variety of studies have shown the beneficial effect of ursodeoxycholic acid in liver disorders. In clinical practice today, UDCA possesses a defined role in treating patients with cholestatic liver diseases. UDCA has been used in clinical practice for more than 20 years and is marketed either as oral tablets or oral capsules with different strengths typically ranging from 50 mg, 150 mg and 300 mg tablets as well as 250 mg capsules.

For some time now, high-dose UDCA treatment has been found to be more effective than and as safe as regular dose treatment in a number of cholestatic hepatic diseases. With the tendency towards the prescription of higher dosages, up to 1 to 2 grams daily, the currently available treatments imply that the patients will need to swallow 4-8 tablets or alternatively 4 to 8 capsules daily and hence presently available dosage strengths are not optimal for this high dose treatment. Moreover, the currently available tablet- and capsule formulations are already bulky and higher dose formulations are becoming very difficult to swallow. Hence, such a dosing regimen could lead to a high rate of patient non-compliance and consequently may lead to failure of therapy.

Furthermore, UDCA is extremely bitter-tasting and causes in most patients esophageal reflux, nausea and/or vomiting. With the solid oral administrations that are currently available, said bitter taste is masked by using capsules and tablets with respectively a gelatine capsule shell or a film coating. Said film coating leads to delay in release of the active ingredient from the formulation.

JP 6209441 discloses a long acting preparation of UDCA conciting of different kinds of granules, viz a rapid release granule agent and a slow release granule agent.

GB 2036558 also discloses a UDCA formulation that releases the active principle over a prolonged period of time that is build up of UDCA that releases immediately and UDCA with a delayed or retarded release.

### SUMMARY OF THE INVENTION

Accordingly, it is an object of the present invention to provide an immediate release formulation of UDCA which allows administration of high daily dosages of UDCA.

It is yet another object of the present invention to provide a formulation which is properly taste masked.

It is a further object of the present invention to provide a formulation that has a proper mouthfeel.

The objects of the present invention were surprisingly met by providing a high UDCA dosage form of between about 400 to 3000 mg UDCA which is prepared as a multiparticulate dosage form. Advantageously the present multiparticulate UDCA dosage form is relatively easier to swallow when compared to the tablets and capsules commercially available. Because of the highly undesirable organoleptic and gastric side effects of UDCA, substantial taste masking of the multiparticulate formulation is a prerequisite. It was found a suitably taste-masked formulation could be provided by a formulation comprising coated particles with UDCA in the particle core. Advantageously the present formulation contains particles with a size of between 100 to 2000 μm thus providing a proper mouthfeel.

It is a further object of the present invention to provide a method for preparing an immediate release multiparticulate formulation of UDCA. This object was met by a method comprising the steps of mixing ursodeoxycholic acid and the at least one pharmaceutically acceptable excipient to form a particle. Subsequently, the particle obtained is coated with a coating agent. Optionally, the coated particles thus obtained are filled into a monodose container.

### DETAILED DESCRIPTION OF THE INVENTION

According to one aspect, the present invention concerns an immediate release oral pharmaceutical formulation in the form of a multiparticulate comprising between 400 or 600 to 3000 mg ursodeoxycholic acid or its pharmaceutically acceptable salt, in a monodose container and comprising a single type of particles that contain a core with said ursodeoxycholic acid, and a coating.

For the purpose of the present invention, the term “immediate release” encompasses that at least 75% of the active ingredient is dissolved within 60 minutes, preferably 75% of the active ingredient is dissolved within 45 minutes, more preferably 70% of the active ingredient is dissolved within 30 minutes (USP Paddle system in phosphate buffer pH 8.0 with 0.1% SDS and operated at 37° C. with a stirring of 100 rpm). The immediate release multiparticulate formulation of the present invention does not comprise an enteric coating, i.e the multiparticulate is not gastric acid resistant and starts dissolving in the stomach.

The term “multiparticulate” encompasses multiple particles which, depending on their shape, may suitably be referred to by a person skilled in the art as a crystal, a granulate, a sphere, a bead, a pellet, a mini-pellet and a mini- or micro-tablet. In the present invention, the multiparticulate comprises multiple particles, said particles comprise the active ingredient and each of said particle being coated. The multiparticulate of the present invention comprises or preferably consists of one type or a single type of particles. One or a single type of particles means that in the present immediate release oral pharmaceutical formulation in the form of a multiparticulate there is no uncoated UDCA present and neither are particles of UDCA present with different types of coatings that would result in a different release profile of the UDCA. Thus one or a single type of particles means that all the UDCA present is coated with the same type of coating that results in immediate release of the UDCA as defined herein.

For the purpose of the present invention, the term “container” encompasses a sachet, a stick pack, a pouch, a bag, a bottle and the like. The container according to the present invention is suited for containing the multiparticulate but is not intended for oral consumption and hence said container should be removed before intake of the formulation. Accordingly, pharmaceutically acceptable capsules such as for example hard gelatin capsules are excluded from the scope of the present invention.

The term “monodose” as used herein is understood to mean the amount of UDCA that is intended to be taken in by a patient in one time.

According to a preferred embodiment, the formulation comprises an amount of 400 mg, 500 mg, 600 mg, 750 mg, 800 mg, 1000 mg, 1200 mg, 1500 mg, 2000 mg, 2500 mg or 3000 mg by weight of ursodeoxycholic acid in a single dose. Said amount is measured as the free acid form of UDCA. Preferably, the formulation is contained in a sachet or in a stick pack. In a more preferred aspect, the amount is between 1000 to 1500 mg ursodeoxycholic acid per sachet.

According to another embodiment, the percentage of UDCA in the particle is between 40 to 90% (w/w), based upon the weight of the particle core. In one embodiment, preferably, the percentage of UDCA in the particle is between 50 to 60%. Also in one embodiment, preferably, the percentage of UDCA in the particle is between 60 to 80%.

According to yet another embodiment, the present invention concerns UDCA particles further comprising at least one pharmaceutically acceptable excipient in the particle core. The term pharmaceutically acceptable excipient as used herein refers to any substance which may be combined with the active ingredient for the preparation of a pharmaceutical formulation and may include a binder, a spheronization aid, a diluent, a disintegration aid, a solubilizer and a flow aid. It is known to the skilled person that an excipient may have multiple functions and hence the below summarized excipients are not to be interpreted as being limited to their indicated function. The used active ingredient, as well as the excipient(s) may suitably be used in micronized form.

Suitable binders for use in the formulation of the invention include, but are not limited to, cellulose and its derivatives, sugar and its derivatives, starch and its derivatives, polyvinyl pyrrolidone, carbomers and waxes.

Suitable spheronization aids for use in the formulation of the invention include, but are not limited to, microcrystalline cellulose, powdered cellulose, kaolin, starch and its derivatives, waxes, crospovidone, pectin or pectinic acid. Spheronization aids may be used either alone or in combination with each other.

According to yet another embodiment, the particle core comprises a pharmaceutically acceptable excipient selected from the group consisting of cellulose and its derivatives, sugar and its derivatives, starch and its derivatives, polyvinyl pyrrolidone, carbomers, waxes or pectin, preferably the pharmaceutically acceptable excipient is a pregelatinized modified starch. More preferably, the pharmaceutically acceptable excipient is crospovidone.

For the present invention, the term cellulose and its derivatives include powdered cellulose, microcrystalline cellulose, hydroxypropyl methylcellulose, hydroxypropyl cellulose, hydroxyethylcellulose, carboxymethyl cellulose, hydroxymethyl ethylcellulose.

For the present invention, the term sugar and its derivatives include sucrose, lactose, mannitol, maltose, maltodextrin, sorbitol.

For the present invention, the term starch and its derivatives may suitably include native starch (derived from maize, potato, rice, tapioca or wheat), pregelatinized starch, high amylose starch, hydroxypropyl starch and hydroxypropyl pregelatinized starch. Preferably, the starch is a pregelatinized modified starch as suitably can be obtained by enzymatic debranching of amylose-rich starch. For example, the pregelatinized modified starch is as disclosed in U.S. Pat. No. 5,281,276, commercially available under the tradename Novelose® 330 or its pharmaceutically grade equivalent (National Starch and Chemical company).

Suitable diluents to include in the formulation of the invention are cellulose and its derivatives, lactose and other suitable sugar derivatives, such as mannitol, sorbitol or calcium phosphates.

Suitable disintegration aids to include in the formulation of the invention are starch and its derivatives, croscarmellose sodium, crospovidone. Solubilizers to include in the formulation of the invention are surfactants, such as sodium dodecyl sulphate, polysorbates, monovalent metal salts of fatty acids, such as sodium stearate, polyethoxylated castor oil derivatives, poloxamers and polyethylene glycols.

Flow aids to include in the formulation of the invention are colloidal silicon dioxide and magnesium stearate.

In yet another embodiment of the present invention, the particle core comprises cellulose and pregelatinized modified starch as pharmaceutically acceptable excipients. Surprisingly, this novel combination of excipients gives pellets with relatively higher degree of sphericity as compared to the individual excipients and its combined use results in a more rapid disintegration time.

The present invention concerns a multiparticulate pharmaceutical formulation comprising a coated particle. Preferably, said coating comprises an agent selected from the group consisting of a water soluble cellulose polymer; an acrylic copolymer; a polyvinyl derivative. Suitably, the water soluble cellulose polymer may be hydroxypropyl methylcellulose, hydroxypropyl cellulose, ethylcellulose and its derivatives. Suitably, the acrylic copolymers may be chosen form the group of buthylmethacrylate-(2-dimethylaminoethyl)methacrylate-methylmethacrylate copolymer (1:2:1); methacrylic acid-methyl methacrylate copolymer (1:1), ethyl acrylate-methyl methacrylate-trimethylammonioethyl methacrylate chloride copolymer (1:2:0.2); ethyl acrylate-methyl methacrylate-trimethylammonioethyl methacrylate chloride copolymer (2:0.1). The polyvinyl derivatives may suitably be polyvinylalcohol. Some of these polymers or combinations thereof are also known under the trade mark Eudragit® (Degussa), Sepifilm® (Seppic), Kollicoat® (BasF Pharma), Opadry 85 (Colorcon), Surelease® (Colorcon).

More preferably, the coating comprises hydroxypropyl methylcellulose and/or buthylmethacrylate-(2-dimethylaminoethyl)methacrylate-methylmethacrylate copolymer (1:2:1) such as for example commercially available under the trade name Eudragit® E (Degussa). In another aspect, the present invention provides a coating, in particular a coating for a pharmaceutical composition, preferably intended for enteral, preferably oral, administration, that comprises a combination of hydroxypropyl methylcellulose and ethylcellulose. In a preferred embodiment of the present invention, the ratio of hydroxypropyl methylcellulose and ethylcellulose is between 1:1.5 to 20 (w/w) Remarkably, this combination results in a direct release coating whereas a controlled release coating is expected.

Suitably, in the coating of the particle also additional compounds such as a plasticizer and/or an anti-adherent may be present. Suitable plasticizers may be triethylcitrate, glyceryl triacetate, polyethylene glycol. Suitable anti-adherents include talc and finely powdered glyceryl monostearate. Surprisingly, the coating of the present invention provides sufficient taste-masking to result in an acceptable taste and at the same time allows for immediate release of the active ingredient.

In another embodiment, the present invention concerns an immediate release oral pharmaceutical formulation in the form of a multiparticulate comprising between 400 or 600 to 3000 mg ursodeoxycholic acid in a monodose container and comprising particle cores comprising said ursodeoxycholic acid and a modified pregelatinized starch and a particle coating comprising hydroxypropyl methyl cellulose.

In yet another embodiment, the multiparticulate formulation comprises particles with a core and a coating wherein the amount of coating is between 1 to 50% (w/w) based on weight of the core. Preferably between 3 to 20%, more preferably it is between 4 to 18%, most preferably it is 5 to 15% (w/w) based on the weight of the core.

In yet another embodiment, the present invention concerns a multiparticulate pharmaceutical formulation further comprising sweeteners and/or flavouring agents. Suitable sweeteners are known in the art and may comprise aspartame, acesulfame potassium, alitame, sucralose, sucrose, saccharose, eriythritol, mannitol, fructose, sorbitol, xylitol, maltitol and saccharin. Suitable flavouring agents are known in the art and may include citric acid, orange flavour, lemon flavour, vanilla flavour.

According to another embodiment, the present invention concerns a formulation further comprising a salivation enhancer. With a salivation enhancer according to the present invention is meant an agent that causes salivation or mouth watering effect. A suitable salivation enhancers may include citric acid.

In another embodiment, the formulation of the present invention contains particles with a size of between 10 or 100 to 1000 or 2000 μm, preferably the size is between 200 to 1200 μm as determined by sieve classification.

In another embodiment, the pharmaceutical formulation is in a solid oral dosage form. The multiparticulate of the present invention can preferably be placed on the tongue and swallowed directly without any addition of liquid. Alternatively, the multiparticulate can be swallowed in the presence of liquid such as water or the like.

In one aspect the present invention concerns a method for preparing a multiparticulate pharmaceutical formulation comprising the steps of:
i. mixing ursodeoxycholic acid and the at least one pharmaceutically acceptable excipient to form a particle;
ii. coating the particle obtained under step i with a coating agent;
iii. optionally, filling the coated particles obtained under step ii into a monodose container.

Preferably, said method of preparation comprises the steps of mixing ursodeoxycholic acid and at least one pharmaceutically acceptable excipient to form a particle core; subsequently coating said particle core, followed by filling the coated particles into a monodose container.

In the particular embodiment wherein the multiparticulate are granules said granulate may be prepared by the process of wet or dry granulation. In the process of wet granulation, UDCA may be mixed with a suitable diluent and wet granulated using a suitable binder solution. The granules are then dried and milled to the required size. In case of dry granulation, UDCA may be mixed with a diluent and alternatively with a dry binder and compacted. The compactate may then be milled to the required particle size. The other alternative for formation of granules or spheres is by the method of extrusion—spheronization.

In the embodiment that the particles of the present invention are pellets, they are prepared according to a method including the steps of mixing ursodeoxycholic acid with at least one pharmaceutically acceptable excipient to form a granulate; extruding the resulting granulate; followed by spheronization of the extrudate to form pellets and then drying the pellets. In yet another preferred embodiment, the pellets or granulate is compressed into micro- or mini-tablets.

Subsequently a layer that comprises a coating agent is applied on the granules, the pellets or the mini- or micro-tablets. Preferably said coating is applied by spray coating according to methods known in the art.

In yet another embodiment of the present invention a flavouring agent is added. Suitably, said flavouring agent may be present in the coating layer or may be applied as separate layer. Preferably, the flavouring agent is applied by mixing the coated particles with dry powder comprising said flavouring agent.

The formulation of the present invention is an immediate release formulation. According to one embodiment, the multiparticulate formulation has an in vitro release profile of at least 75% released within 60 minutes. Preferably 75% is released within 45 minutes. More preferably, 70% released within 30 minutes when measured in a model system using USP Paddle system in phosphate buffer pH 8.0 and 0.1% SDS, operated at 37° C. with a stirring of 100 rpm. In a preferred embodiment of the present invention, 80% is released within 30 minutes. More preferably, 90% is released within 30 minutes. Most preferably, 100% of the UDCA is released within 20 minutes.

The multiparticulate pharmaceutical formulation of the present invention is packed in a monodose container. Said monodose container contains multiple particles comprising UDCA corresponding to a single dose. Suitable containers include a sachet, a stick pack, and likely packaging material known to people skilled in the art. The multiparticulate formulation of the present invention may preferably be administered to the patient without the need for co-administering water or another fluid. The multiparticulate of the present invention has a good mouthfeel and is easy to swallow. Moreover, the multiparticulate formulation of the present invention comprises particles wherein the bitter taste of UDCA is masked to an extent that is acceptable to patients.

It is yet another object of the present invention to provide the use of an immediate release formulation in the form of multiparticulate in a monodose container comprising between 400 or 500 or 600 to 3000 mg ursodeoxycholic acid for the preparation of a medicament for the treatment of primary biliary cirrhosis.

The present invention is further illustrated by the following example which should not be interpreted as limiting the scope of the invention.

### Example 1

### Preparation of UDCA Pellet Formulation

### Manufacturing of Pellets

Formulation (batch size: 500 g):

| | |
|---|---|
| API (UDCA) | 50% (w/w, dry mass) |
| HPMC (Methocel ®E15 LV) | 5% (w/w, dry mass) |
| UNI-PURE ® EX starch | 45% (w/w, dry mass) |
| Water | 52% (based on solid content) |

A batch of 500 gram pellets is prepared by extrusion-spheronization. 250 grams of UDCA API, 25 grams of HPMC (Methocel E15) and 225 grams of Unipure EX-starch (National Starch and Chemical Company; also know as Novelose® 330) were weighed, transferred and mixed in a high shear mixer for 3 minutes. Water was added (260 ml) and a homogeneous wet mass was prepared by blending for 5 minutes.

The wetted mass was transferred into the feeding chamber of the extruder and the mixture extrudated. Extrusion screen diameter: 600 μm. The extrudate was transferred directly into the spheronizer. Spheronizing speed 800 rpm and spheronization was stopped after 3 minutes. After spheronization the pellets were dried in the oven.

Before coating, pellets with a size lower than 300 μm and above 800 μm were excluded from the material by sieving. 400 grams of the uncoated pellets could be used for coating.

### Manufacturing of Coated Pellets

An Opadry II (Colorcon) coating suspension was prepared containing vanillin. First the vanillin was dispersed in water with an ultraturrax. This water was then added to the Opadry II powder and the coating solution was prepared by continuous stirring for 45 minutes. The solids content of the coating solution was 15%.

400 grams of sieved pellets were coated with the Opadry II/vanillin coating suspension in a fluidized bed. The product temperature was kept at 40-45° C., the spray gun was operated with a nozzle of 0.8 mm and a atomizing air pressure of 2 bars. The spray rate was 4 g/min. When a coating weight gain of 5% solids was reached, the coating was stopped.

The resulting coated pellets have an acceptable taste and the dissolution tests show that the release of active ingredient is more than 80% within half an hour.

### Example 2

### Preparation of UDCA Pellet Formulation

### Manufacturing of Pellets

Formulation (batch size: 500 g):

| | |
|---|---|
| API (UDCA) | 69% (w/w, dry mass) |
| Crospovidone (BASF) | 20% (w/w, dry mass) |
| HPMC | 2% (w/w, dry mass) |
| Microcrystalline cellulose | 5% (w/w, dry mass) |
| Polyethylene glycol | 4% (w/w, dry mass) |
| Water | 35% (based on solid content) |

A batch of 500 gram pellets is prepared by extrusion-spheronization as described in example 1.

### Manufacturing of Coated Pellets

A mixture of Opadry II (Colorcon) coating suspension (20%) and Surelease® (Colorcon) coating suspension (80%) was prepared.

400 grams of sieved pellets were coated according to the method described in example 1. When a coating weight gain of 4% solids was reached, the coating was stopped.

Multiple coated pellets, corresponding to 1000 mg active, were packed in a sachet.

The resulting coated pellets have an acceptable taste and the dissolution tests show that the release of active ingredient is more than 75% within 60 minutes

1. An immediate release oral pharmaceutical formulation in the form of a multiparticulate comprising between 400 to 3000 mg ursodeoxycholic acid or its pharmaceutically acceptable salt, in a unit dose container and comprising a single type of particle that contains (i) a core with said ursodeoxycholic acid, and (ii) a coating.

2. The formulation according to claim 1, further comprising at least one pharmaceutically acceptable excipient in the particle core that is selected from the group consisting of cellulose, a cellulose derivative, a sugar, a sugar derivative, a starch, a starch derivative, polyvinyl pyrrolidone, and a carbomer or wax.

3. The formulation according to claim 1, wherein the amount of ursodeoxycholic acid in each particle is between 40 to 90% (w/w) based on the total weight of the particle core.

4. The formulation according to claim 1 wherein the coating comprises an agent selected from the group consisting of a water soluble cellulose polymer; an acrylic copolymer; and a polyvinyl derivative.

5. The formulation according to claim 1, wherein the size of the particles is between 10 to 2000 μm.

6. The formulation according to claim 1, wherein the particle coating is between 1% and 50% (w/w) of the weight of the particle core.

7. The immediate release formulation according to claim 1, wherein (i) the particle cores comprise ursodeoxycholic acid and crospovidone and (ii) the particle coating comprises hydroxypropyl methyl cellulose.

8. A method for preparing a multiparticulate pharmaceutical formulation comprising the steps of:
(i) mixing ursodeoxycholic acid and the at least one pharmaceutically acceptable excipient to form a particle;
(ii) coating the particle obtained in step (i) with a coating agent;
(iii) optionally, filling the coated particles obtained in step (ii) into a unit dose container.

9. The method according to claim 8, wherein the pharmaceutically acceptable excipient is crospovidone, and the coating agent is hydroxypropyl methylcellulose and/or ethylcellulose.

10. A method of treating a subject with primary biliary cirrhosis, comprising administering to the subject an immediate release formulation in the form of a multiparticulate in a unit dose container comprising between 400 to 3000 mg ursodeoxycholic acid or a pharmaceutically acceptable salt thereof.

11. The formulation according to claim 2 wherein the pharmaceutically acceptable excipient is crospovidone.

12. The formulation according to claim 4 wherein the agent is hydroxypropyl methylcellulose and/or ethylcellulose.

13. The formulation according to claim 6, wherein the particle coating is between 3% and 20% (w/w) of the weight of the particle core.

14. The formulation according to claim 6, wherein the particle coating is between 5% and 15% (w/w) of the weight of the particle core.
